(19) United States
(12) Plant Patent Application Publication
(10) Pub. No.: US 2014/0338082 P1
(43) Pub. Date: Nov. 13, 2014

(54) Chamelaucium plant named 'FIRST MAYA'

(50) ;

(71) Applicant: Nitzan Nir, Kfar Hess (IL)

(72) Inventor: Nitzan Nir, Kfar Hess (IL)

(21) Appl. No.: 13/986,495

(22) Filed: May 7, 2013

(51) Int. Cl. A01H 5/00

(52) U.S. Cl. PLT/229

(57) ABSTRACT

A new and distinct Chamelaucium cultivar named ‘FIRST MAYA’ is disclosed, characterized by medium sized, creamy white colored flowers. The new variety is very early blooming. The new variety is a Chamelaucium, normally producing cut flower stems, or as a garden or container plant.

### LATIN NAME OF THE GENUS AND SPECIES

Chamelaucium uncinatum×megalopetalum

### VARIETY DENOMINATION

‘FIRST MAYA’

### BACKGROUND OF THE INVENTION

The new Chamelaucium cultivar is a product of chance discovery by the inventor, Nitzan Nir, in Kfar Hess, Israel. The new variety was selected as a naturally occurring whole plant mutation of the variety Chamelaucium uncinatum×megalopetalum ‘Early Maya’, unpatented. ‘First Maya’ was selected during October of 2009, at a commercial nursery in Kfar Hess, Israel.

Asexual reproduction of the new cultivar has been performed by vegetative cuttings. This was first performed at a commercial nursery in Kfar Hess, Israel in December of 2010 and has shown that the unique features of this cultivar are stable and reproduced true to type in successive generations.

### SUMMARY OF THE INVENTION

The cultivar ‘FIRST MAYA’ has not been observed under all possible environmental conditions. The phenotype may vary somewhat with variations in environment such as temperature, day length, and light intensity, without, however, any variance in genotype.

The following traits have been repeatedly observed and are determined to be the unique characteristics of ‘FIRST MAYA’. These characteristics in combination distinguish ‘FIRST MAYA’ as a new and distinct Chamelaucium cultivar:
1. Very early blooming season beginning in September in the Northern Hemisphere.
2. Creamy white flower.
3. Medium flower size.
4. Pink flower bud.

### Parent Comparison

Plants of the new cultivar ‘FIRST MAYA’ are similar to plants of the parent, Chamelaucium uncinatum×megalopetalum ‘Early Maya’ in most horticultural characteristics, however, plants of the new cultivar ‘FIRST MAYA’ produce larger flowers one month earlier than the parent.

### Commercial Comparison

Plants of the new cultivar ‘FIRST MAYA’ are comparable to the variety Chamelaucium ‘Blondie’, U.S. Plant Pat. No. 9,966. The two Chamelaucium varieties are similar in most horticultural characteristics; however, the new variety ‘FIRST MAYA’ begins flowering earlier than ‘Blondie’.

Plants of the new cultivar ‘FIRST MAYA’ can also be compared to the commercial variety Chamelaucium ‘Guy’, unpatented. These varieties are similar in most horticultural characteristics; however ‘FIRST MAYA’ flowers earlier than ‘Guy’.

### BRIEF DESCRIPTION OF THE PHOTOGRAPH

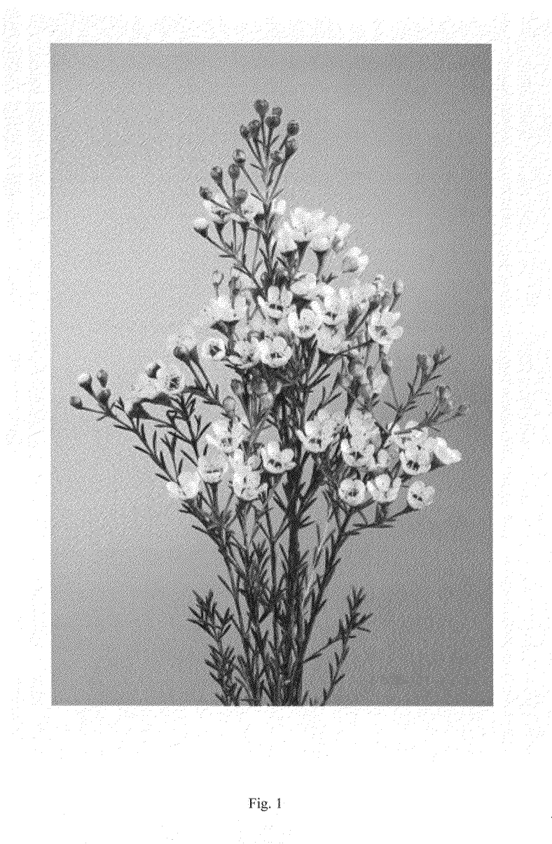

The accompanying photograph in FIG. 1 illustrates in full color a close up of typical flowers and foliage of ‘FIRST MAYA’. The photograph was taken using conventional techniques and although colors may appear different from actual colors due to light reflectance it is as accurate as possible by conventional photographic techniques.

### DETAILED BOTANICAL DESCRIPTION

In the following description, color references are made to The Royal Horticultural Society Mini Colour Chart 2005 except where general terms of ordinary dictionary significance are used. The following observations and measurements describe ‘FIRST MAYA’ plants grown outdoors in Kfar Hess, Israel. The growing temperature ranged from 15° C. to 35° C. during the day and from −2° C. to 35° C. during the night. General light conditions are bright, normal sunlight. Measurements and numerical values represent averages of typical plant types.
Botanical classification: Chamelaucium uncinatum×megalopetalum ‘FIRST MAYA’
Age of plant described: At least 2 months old from a rooted cutting.
Typical flowering stems harvested per plant: 100 to 120 during the course of the season.
Vase life of flowers: Two weeks
Propagation:
Time to produce a rooted plantlet: 30 to 60 days at approximately 24° C.
Root description: Fibrous.
Plant:
Growth habit: Upright plant produces multiple stems from a pinch, or after harvesting stems.
Height: 150 cm.
Plant spread: 50 cm.
Growth rate: Rapid
Branching characteristics: Moderate branching occurs after pinching.
Length of primary lateral branches: 60 cm on average.
Diameter of lateral branches: 0.3 cm.
Quantity of primary lateral branches: 100 to 120. These are the harvested flowering stems.
Characteristics of primary lateral branches:
   Form.—Round.
   Diameter.—0.4 cm.
   Color.—Near RHS Yellow-Green 152B over Grey-Grown 199A.
   Texture.—Smooth.
   Strength.—Stems brittle.
Internode length: Range from 0.5 to 6 cm.
Foliage:
Leaf:
   Arrangement.—Opposite.
   Quantity.13 Approximately 10 leaves, in the 10-15 cm section from the top of the branch.
   Average length.—0.8 cm.
   Average width.—0.1 cm.
   Shape of blade.—Needle.
   Apex.—Acute.
   Base.—Attenuate.
   Margin.—Entire.
   Texture.—Glabrous all surfaces.
   Pubescence.—None.
   Aspect.—Straight, occurring at approximately a 15 to 30 degree angle.
   Color.—Young foliage: RHS Yellow-Green 146C Mature foliage: RHS Yellow-Green 146B Venation: Indistinguishable from foliage.
   Petiole.—Not present.
Flower:
Natural flowering season: Plants planted during April through October begin flowering September through January, without a juvenility period. Plants flower continuously under short day conditions.
Natural flowering season: Short days.
Days to flowering from rooted cutting: Approximately 60 days.
Inflorescence type: Panicle.
Individual flower type: Single, 5 petals with hypanthium.
Persistent or self-cleaning: Self-Cleaning
Bud:
   Shape.—Spherical.
   Length.—0.5 cm.
   Diameter.—0.5 cm.
   Color.—Near RHS Red 50A.
Inflorescence size:
   Diameter.—Average 12 cm.
   Length.—Average 22 cm.
Flower size:
   Diameter.—Average 1.2 cm.
   Length.—Average 1.3 cm.
Corolla:
Petals:
   Arrangement.—Rotate, not overlapping.
   Length.—Average 0.7 cm.
   Diameter.—Average 0.7 cm.
   Quantity.—5.
   Texture.—Smooth.
   Apex.—Obtuse rounded.
   Base.—Fused into a hypanthium.
Color:
   When opening.—Upper surface: Near RHS White 155B Lower surface: Near RHS White 155B.
   Fully opened.—Upper surface: Near RHS White 155B Lower surface: Near RHS White 155B.
   Aging.—Upper surface: Near RHS White 155C Lower surface: Near RHS White 155C.
Hypanthium:
   Diameter.—Average 0.7 cm.
   Length.—Average 0.4 cm.
Color:
   When opening.—Inner surface: Near RHS Green-White 155D, blotched Red 53B Outer surface: Near RHS White 155B.
   Fully opened.—Inner surface: Near RHS Green-White 155D, blotched Red 53B Outer surface: Near RHS White 155B.
   Aging.—Inner surface: Near RHS Green-White 155D, blotched Red 53B Outer surface: Near RHS White 155B.
Calyx/sepals:
   Quantity per flower.—Fused into a single, conical structure.
   Length.—0.4 cm.
   Width.—0.7 cm.
   Margin.—Entire.
   Texture.—Smooth.
Peduncle: Peduncle consists of the plant stem.
Pedicel:
   Length.—0.6 cm.
   Diameter.—0.1 cm.
Fragrance: None.
Reproductive organs:
Stamens:
   Number.—10.
   Filament length.—1 mm.
Anthers:
   Shape.—Oval.
   Length.—0.25-0.5 mm.
Pollen: Not observed to date.
Pistil:
   Number.—1.
   Length.—0.5 cm.
   Length.—0.8 cm.
   Color.—Near RHS White 155A.
   Stigma.—Shape: Round.
Other characteristics:
Seeds and fruits: Single flower produces one fruit. Fruits are not fertile.
Disease/pest resistance: Neither resistance nor susceptibility to the normal diseases and pests of Chamelaucium have been observed to date.
Temperature tolerance: From −2° C. to 35° C.
Drought tolerance: Very good tolerance for drought.

What is claimed is:

1. A new and distinct cultivar of Chamelaucium plant named ‘FIRST MAYA’ as herein illustrated and described.
